(19) United States
(12) Patent Application Publication
(10) Pub. No.: US 2023/0414419 A1
(43) Pub. Date: Dec. 28, 2023

(54) Undergarment Device with a Replaceable Section

(71) Applicant: Marsha Jones, Chicago, IL (US)

(72) Inventor: Marsha Jones, Chicago, IL (US)

(21) Appl. No.: 18/337,465

(22) Filed: Jun. 20, 2023

Related U.S. Application Data
(60) Provisional application No. 63/354,316, filed on Jun. 22, 2022.

(51) Int. Cl. A61F 13/70; A61F 13/74; A41B 9/00

(52) U.S. Cl. CPC A61F 13/70; A61F 13/74; A41B 9/001

(57) ABSTRACT

An undergarment device with a replaceable section is provided. The device is comprised of a body that receives at least one replaceable section that covers the crotch of the user during use. The section attaches to a front area and a rear area of the body via fasteners, such that the section can be removed if it becomes soiled with bodily fluid. Then, the section can be washed and/or replaced with a new section.

### CROSS-REFERENCE TO RELATED APPLICATION

The present application claims priority to, and the benefit of, U.S. Provisional Application No. 63/354,316, which was filed on Jun. 22, 2022, and is incorporated herein by reference in its entirety.

### FIELD OF THE INVENTION

The present invention relates generally to the field of undergarments. More specifically, the present invention relates to an undergarment device with a replaceable section that provides an undergarment with a removable section that can be easily detached from the undergarment if said section becomes soiled with bodily fluids. Then, the soiled section can be washed or replaced with a new, unsoiled section. Accordingly, the present disclosure makes specific reference thereto. Nonetheless, it is to be appreciated that aspects of the present invention are also equally applicable to other like applications, devices, and methods of manufacture.

### BACKGROUND

Bodily fluid leakage onto an undergarment during a woman's menstrual cycle is an occurrence that many women are familiar with. However, dealing with stained undergarments can be quite inconvenient and potentially frustrating. The importance of addressing these stains promptly is paramount if one wishes to maintain the longevity and cleanliness of their undergarments. When a menstrual stain occurs, it is crucial to take quick action to prevent the stain from setting and becoming more challenging to remove. As a result, many individuals prefer to immediately wash their stained undergarments in order to tackle the issue head-on. By promptly rinsing and treating the stain, one can significantly increase the chances of successfully removing it. This method allows for the possibility of salvaging the undergarment, thereby saving money and avoiding the hassle of purchasing new undergarments.

However, there are instances where immediate washing might not be possible or convenient. This could be due to various factors, such as being away from home, lacking access to laundry facilities, or simply being caught off guard by the sudden appearance of a stain. In such cases, the option of discarding the stained undergarment and purchasing new ones may seem like a viable solution.

Therefore, there exists a long-felt need in the art for an improved undergarment. There also exists a long-felt need in the art for an undergarment device with a replaceable section that prevents an undergarment from being ruined/stained by bodily fluids. Further, there exists a long-felt need in the art for an undergarment device with a replaceable section that reduces the cost associated with buying new undergarments due to the soiling/staining of undergarments by bodily fluids. In addition, there exists a long-felt need in the art for an undergarment device with a replaceable section that prevents an undergarment from being ruined/stained by bodily fluids by providing a removable section of the undergarment that can be easily and quickly detached.

The subject matter disclosed and claimed herein, in one embodiment thereof, comprises an undergarment device with a replaceable section. The device is comprised of a body that receives at least one replaceable section that covers the crotch of the user during use. The section attaches to a front area and a rear area of the body via fasteners, such that the section can be removed if it becomes soiled with bodily fluid. Then, the section can be washed and/or replaced with a new section.

In this manner, the undergarment device with a replaceable section of the present invention accomplishes all the foregoing objectives and provides an improved undergarment. More specifically, the device provides an undergarment that cannot be easily ruined/stain by bodily fluids. As such, the device reduces the cost associated with buying new undergarments due to the soiling/staining of undergarments by bodily fluids. In addition, the removable section of the undergarment can be easily and quickly detached and re-attached for ease of use.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an undergarment device with a replaceable section. The device is comprised of an undergarment body with at least one replaceable section that can be removed from the body when soiled. Said area can then be replaced with a new, clean replaceable section and washed or disposed of. The body may be shaped like any style, size, and shape of women's underwear known in the art. The body may also be made from any material (or combination of materials) known in the art. The body is comprised of a top opening. The top opening may be comprised of at least one elastic waistband in one embodiment that secures the body around the waist of the user. The body is also comprised of a first leg opening and a second leg opening.

The body is also comprised of a front area and a rear area. The front area is comprised of a bottom edge having at least one fastener. The fastener attaches to the fastener of a first edge of a replaceable section. Similarly, the rear area is comprised of a bottom edge having at least one fastener. The fastener attaches to the fastener of a second edge of a replaceable section. In this manner, the replaceable section can be removed and easily washed or discarded (and replaced with a new replaceable section) if the area is stained with bodily fluids. The fasteners may be any type of reciprocating fasteners known in the art. However, in the preferred embodiment, the fasteners are a combination of clip and loop fasteners. In one embodiment, the edges of the front area and rear area are comprised of a padding positioned behind the fasteners for comfort purposes.

The present invention is also comprised of a method of using the device. First, a device is provided comprised of a body comprised of a front area with an edge comprised of a first fastener, a rear area comprised of an edge with a second fastener, and a replaceable section comprised of a first edge comprised of a third fastener and a second edge comprised of a second fastener. Then, the fastener of the first edge of the replaceable section is attached to the fastener of the front area. Then, the fastener of the second edge of the replaceable section is attached to the fastener of the rear area. If the replaceable section becomes soiled and/or stained with bodily fluid, the fasteners of the replaceable section can be removed from the fasteners of the front area and rear area. The replaceable section can then be washed and re-attached to the fasteners or thrown away wherein a new replaceable section is attached to the fasteners.

Accordingly, the undergarment device with a replaceable section of the present invention is particularly advantageous as it provides an improved undergarment. More specifically, the device provides an undergarment that cannot be easily ruined/stained by bodily fluids. As such, the device reduces the cost associated with buying new undergarments due to the soiling/staining of previous undergarments by bodily fluids. In addition, the removable section of the undergarment can be easily and quickly detached and re-attached for ease of use. In this manner, the undergarment device with a replaceable section overcomes the limitations of existing undergarments known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of one potential embodiment of an undergarment device with a replaceable section of the present invention in accordance with the disclosed architecture;

FIG. 2 illustrates a rear view of one potential embodiment of an undergarment device with a replaceable section of the present invention in accordance with the disclosed architecture;

FIG. 3 illustrates a perspective view of one potential embodiment of an undergarment device with a replaceable section of the present invention while the replaceable area is unattached in accordance with the disclosed architecture; and

FIG. 4 illustrates a flowchart of a method of using one potential embodiment of an undergarment device with a replaceable section of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved undergarment. There also exists a long-felt need in the art for an undergarment device with a replaceable section that prevents an undergarment from being ruined/stained by bodily fluids. Further, there exists a long-felt need in the art for an undergarment device with a replaceable section that reduces the cost associated with buying new undergarments due to the soiling/staining of undergarments by bodily fluids. In addition, there exists a long-felt need in the art for an undergarment device with a replaceable section that prevents an undergarment from being ruined/stained by bodily fluids by providing a removable section of the undergarment that can be easily and quickly detached.

The present invention, in one exemplary embodiment, is comprised of an undergarment device with a replaceable section. The device is comprised of an undergarment body with at least one replaceable section that can be removed from the body when soiled. Said area can then be replaced with a new, clean replaceable section and washed or disposed of.

The body may be shaped like any style, size, and shape of women's underwear known in the art and may be made from any material (or combination of materials) known in the art. The body is comprised of a top opening. The top opening may be comprised of at least one elastic waistband in one embodiment that secures the body around the waist of the user. The body is also comprised of a first leg opening and a second leg opening.

The body is also comprised of a front area and a rear area. The front area is comprised of a bottom edge having at least one fastener. The fastener attaches to the fastener of a first edge of a replaceable section. Similarly, the rear area is comprised of a bottom edge having at least one fastener. The fastener attaches to the fastener of a second edge of a replaceable section. In this manner, the replaceable section can be removed and easily washed or discarded (and replaced with a new replaceable section) if the area is stained with bodily fluids. The fasteners may be any type of reciprocating fasteners known in the art. However, in the preferred embodiment the fasteners are a combination of clip and loop fasteners. In one embodiment, the edges of the front area and rear area are comprised of a padding positioned behind the fasteners for comfort purposes.

The present invention is also comprised of a method of using the device. First, a device is provided comprised of a body comprised of a front area with an edge comprised of a first fastener, a rear area comprised of an edge with a second fastener, and a replaceable section comprised of a first edge comprised of a third fastener and a second edge comprised of a second fastener. Then, the fastener of the first edge of the replaceable section is attached to the fastener of the front area. Then, the fastener of the second edge of the replaceable section is attached to the fastener of the rear area. If the replaceable section 140 becomes soiled and/or stained with bodily fluid, the fasteners of the replaceable section can be removed from the fasteners of the front area and rear area. The replaceable section can then be washed and re-attached to the fasteners or thrown away, wherein a new replaceable section is attached to the fasteners.

Accordingly, the undergarment device with a replaceable section of the present invention is particularly advantageous as it provides an improved undergarment. More specifically, the device provides an undergarment that cannot be easily ruined/stain by bodily fluids. As such, the device reduces the cost associated with buying new undergarments due to the soiling/staining of previous undergarments by bodily fluids. In addition, the removable section of the undergarment can be easily and quickly detached and re-attached for ease of use. In this manner, the undergarment device with a replaceable section overcomes the limitations of existing undergarments known in the art.

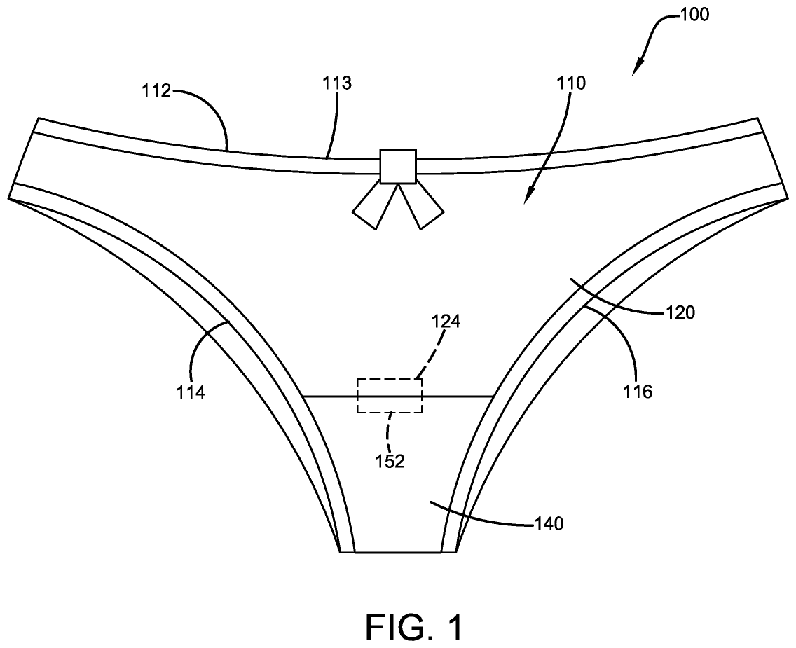

Referring initially to the drawings, FIG. 1 illustrates a front view of one potential embodiment of an undergarment device with a replaceable section 100 of the present invention in accordance with the disclosed architecture. The device 100 is comprised of an undergarment body 110 with at least one replaceable section 140 that can be removed from the body 110 when soiled and replaced with a new, clean replaceable section 140. The body 110 may be shaped like any style, size, and shape of women's underwear known in the art such as, but not limited to, a G-string, a thong, a tanga, a cheeky, a bikini, a hipster, a boy brief, a boy shorts, a boxer, a hi-wasted, a T-string, a control brief, a maxi, a Brazilian, a classic, a pantaloon, a string, or any other type known in the art. The body 110 may be made from any material (or combination of materials) known in the art such as, but not limited to, cotton, lace, latex, leather, lycra, mesh, nylon, PVC, polyester, rawhide, satin, silk, etc.

The body 110 is comprised of a top opening 112. The top opening 112 may be comprised of at least one elastic waistband 113 in one embodiment that secures the body 110 around the waist of the user. The body 110 is also comprised of a first leg opening 114 and a second leg opening 116.

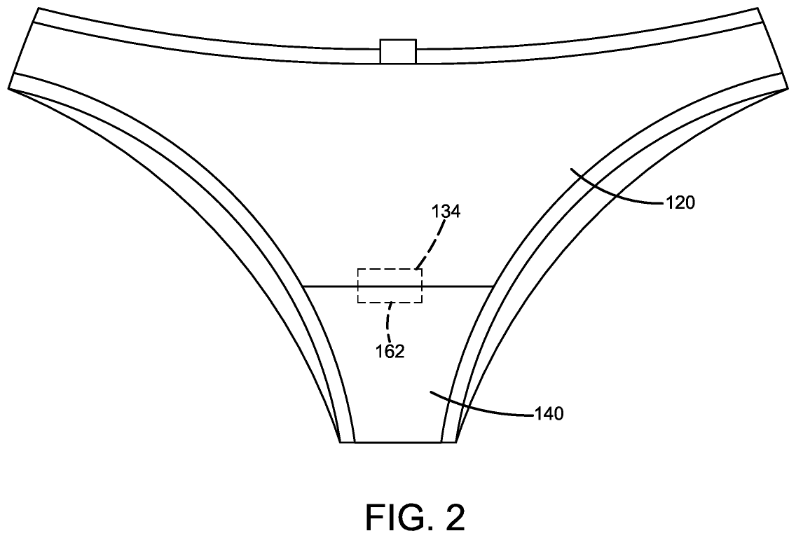

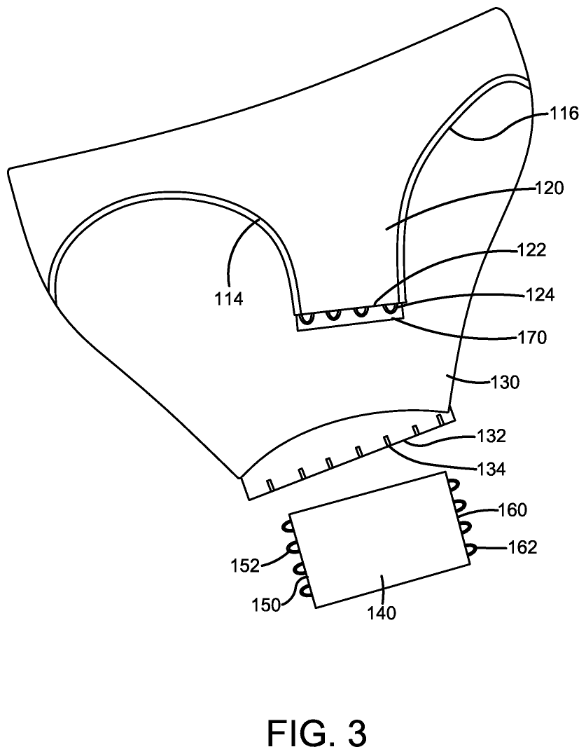

The body 110 is comprised of a front area 120 and a rear area 130, as seen in FIG. 1 and FIG. 2. The front area 120 is comprised of a bottom edge 122. The bottom edge 122 is comprised of at least one fastener 124. The fastener 124 attaches to the fastener 152 of a first edge 150 of a replaceable section 140, as seen in FIG. 3.

The rear area 130 is comprised of a bottom edge 132. The bottom edge 132 is comprised of at least one fastener 134. The fastener 134 attaches to the fastener 162 of a second edge 160 of a replaceable section 140, as seen in FIG. 3.

In this manner, the section 140 can be removed and easily washed or discarded (and replaced with a new replaceable section 140) if the section 140 is stained with bodily fluids. The fasteners 124,134,152,162 may be any type of reciprocating fasteners known in the art. This includes fasteners such as, but not limited to, hook and loop, snap button, magnet, adhesive, zipper, button and loop, etc. However, in the preferred embodiment the fasteners 124,134,152,162 are a combination of clip and loop fasteners.

In one embodiment, the replaceable section 140 is made from the same material as the rest of the body 110. In another embodiment, the replaceable area 140 is made from an absorbent material to absorb bodily fluids and ensure said fluids do not leak through the device 100 and onto the shorts, pants, dress, etc., of the user.

In one embodiment, the edges 122,132 of the front area 120 and rear area 130 are comprised of a padding 170. The padding 170 is positioned behind the fasteners 124,134 for comfort purposes.

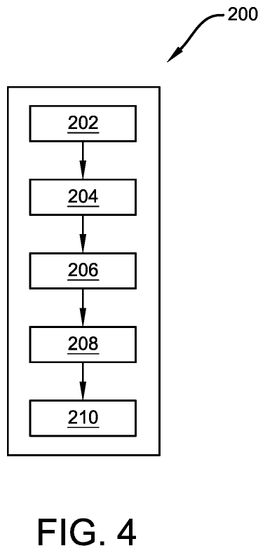

The present invention is also comprised of a method of using 200 the device 100, as seen in FIG. 4. First, a device 100 is provided comprised of a body 110 comprised of a front area 120 with an edge 122 comprised of a first fastener 124, a rear area 130 comprised of an edge 132 with a second fastener 134, and a replaceable section 140 comprised of a first edge 150 comprised of a third fastener 152 and a second edge 160 comprised of a second fastener 162 [Step 202]. Then, the fastener 152 of the first edge 150 of the replaceable section 140 is attached to the fastener 124 of the front area 120 [Step 204]. Then, the fastener 162 of the second edge 160 of the replaceable section 140 is attached to the fastener 134 of the rear area 130 [Step 206]. If the replaceable section 140 becomes soiled and/or stained with bodily fluid, the fasteners 152,162 of the replaceable section can be removed from the fasteners 124,134 of the front area 120 and rear area 130 [Step 208]. The replaceable section 140 can then be washed and re-attached to the fasteners 124,134 or thrown away, wherein a new replaceable section 140 is attached to the fasteners 124,134 [Step 210].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “undergarment device with a replaceable section” and “device” are interchangeable and refer to the undergarment device with a replaceable section 100 of the present invention.

Notwithstanding the foregoing, the undergarment device with a replaceable section 100 of the present invention and its various components can be of any suitable size and configuration as is known in the art without affecting the overall concept of the invention, provided that they accomplish the above-stated objectives. One of ordinary skill in the art will appreciate that the size, configuration, and material of the undergarment device with a replaceable section 100 as shown in the FIGS. are for illustrative purposes only, and that many other sizes and shapes of the undergarment device with a replaceable section 100 are well within the scope of the present disclosure. Although the dimensions of the undergarment device with a replaceable section 100 are important design parameters for user convenience, the undergarment device with a replaceable section 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An undergarment device with a replaceable section comprising:
a body comprised of a front area, a rear area, a top opening, a first leg opening, and a second leg opening;
a first fastener positioned on a bottom edge of the front area;
a second fastener positioned on a bottom edge of the rear area; and
a replaceable section comprised of a first edge comprised of a third fastener and a second edge comprised of a fourth fastener.

2. An undergarment device with a replaceable section of claim 1, wherein the body is comprised of a G-string, a thong, a tanga, a cheeky, a bikini, a hipster, a boy brief, a boy shorts, a boxer, a hi-wasted, a T-string, a control brief, a maxi, a Brazilian, a classic, a pantaloon, or a string body.

3. An undergarment device with a replaceable section of claim 1, wherein the first fastener and the third fastener are comprised of a pair of reciprocating fasteners.

4. An undergarment device with a replaceable section of claim 3, wherein the first fastener and the third fastener are comprised of a hook and loop fastener, a snap button fastener, a magnet fastener, an adhesive fastener, a zipper fastener, a button and loop fastener, or a clip and loop fastener.

5. An undergarment device with a replaceable section of claim 1, wherein the second fastener and the fourth fastener are comprised of a pair of reciprocating fasteners.

6. An undergarment device with a replaceable section of claim 5, wherein the second fastener and the fourth fastener are comprised of a hook and loop fastener, a snap button fastener, a magnet fastener, an adhesive fastener, a zipper fastener, a button and loop fastener, or a clip and loop fastener.

7. An undergarment device with a replaceable section of claim 1, wherein the replaceable section is comprised of an absorbent material.

8. An undergarment device with a replaceable section of claim 1, wherein the top opening is comprised of an elastic waistband.

9. An undergarment device with a replaceable section comprising:
a body comprised of a front area, a rear area, a top opening, a first leg opening, and a second leg opening;
a first fastener positioned on a bottom edge of the front area;
a second fastener positioned on a bottom edge of the rear area;
a replaceable section comprised of a first edge comprised of a third fastener and a second edge comprised of a fourth fastener; and
a padding.

10. An undergarment device with a replaceable section of claim 9, wherein the body is comprised of a G-string, a thong, a tanga, a cheeky, a bikini, a hipster, a boy brief, a boy shorts, a boxer, a hi-wasted, a T-string, a control brief, a maxi, a Brazilian, a classic, a pantaloon, or a string body.

11. An undergarment device with a replaceable section of claim 9, wherein the first fastener and the third fastener are comprised of a pair of reciprocating fasteners.

12. An undergarment device with a replaceable section of claim 11, wherein the first fastener and the third fastener are comprised of a hook and loop fastener, a snap button fastener, a magnet fastener, an adhesive fastener, a zipper fastener, a button and loop fastener, or a clip and loop fastener.

13. An undergarment device with a replaceable section of claim 9, wherein the second fastener and the fourth fastener are comprised of a pair of reciprocating fasteners.

14. An undergarment device with a replaceable section of claim 13, wherein the second fastener and the fourth fastener are comprised of a hook and loop fastener, a snap button fastener, a magnet fastener, an adhesive fastener, a zipper fastener, a button and loop fastener, or a clip and loop fastener.

15. An undergarment device with a replaceable section of claim 9, wherein the replaceable section is comprised of an absorbent material.

16. An undergarment device with a replaceable section of claim 9, wherein the padding is positioned underneath the first fastener.

17. An undergarment device with a replaceable section of claim 9, wherein the padding is positioned underneath the second fastener.

18. An undergarment device with a replaceable section of claim 9, wherein the top opening is comprised of an elastic waistband.

19. A method of using an undergarment device with a replaceable section, the method comprising the following steps:
providing an undergarment device with a replaceable section comprised of a body comprised of a front area with a first edge comprised of a first fastener, a rear area comprised of a second edge with a second fastener, and a first replaceable section comprised of a third edge comprised of a third fastener and a fourth edge comprised of a fourth fastener;
attaching the first fastener to the third fastener of the first replaceable section;
attaching the second fastener to the fourth fastener of the first replaceable section;
removing the first fastener from the third fastener if the first replaceable section becomes stained or soiled;
removing the second fastener from the fourth fastener if the first replaceable section becomes stained or soiled;
providing a second replaceable section comprised of a third edge comprised of a third fastener and a fourth edge comprised of a fourth fastener;
attaching the first fastener to the third fastener of the second replaceable section; and
attaching the second fastener to the fourth fastener of the second replaceable section.

20. An undergarment device with a replaceable section of claim 19, wherein the first fastener, the second fastener, the third fastener, the fourth fastener are comprised of reciprocating fasteners.
